(19) United States
(12) Patent Application Publication
(10) Pub. No.: US 2001/0031924 A1
(43) Pub. Date: Oct. 18, 2001

(54) Small ultrasound transducers

(76) Inventor: James B. Seward, Rochester, MN (US)

Correspondence Address: MERCHANT & GOULD PC, P.O. BOX 2903, MINNEAPOLIS, MN, 55402-0903, US

(21) Appl. No.: 09/797,397

(22) Filed: Mar. 1, 2001

Related U.S. Application Data
(60) Provisional application No. 60/186,395, filed on Mar. 2, 2000.

(51) Int. Cl. A61B 8/14

(52) U.S. Cl. 600/459

(57) ABSTRACT

A miniaturized ultrasound transducer (e.g. less than 4 mm×4 mm×10 mm) is provided and is operable in small spaces, such as a blood-vessel-size type of underfluid structure. The ultrasound transducer may be mounted onto or incorporated into a holding device to be easily manipulated. The ultrasound transducer communicates with a processing unit via an electrical wire or cable or wirelessly.

### RELATED APPLICATION

This application claims the benefit of Provisional Application, U.S. Ser. No. 60/186,395, filed on Mar. 2, 2000, entitled “SMALL ULTRASOUND TRANSDUCERS”, by James B. Seward, which is incorporated herewith by reference.

### FIELD OF THE INVENTION

The present invention relates to ultrasound transducers, and more particularly, to small or miniaturized ultrasound transducers.

### BACKGROUND OF THE INVENTION

Ultrasound is a ubiquitous technology capable of obtaining images, assessing functions, measuring hemodynamics, characterizing tissues, visualizing fluid flows, etc. One of the major attributes of ultrasound is its safety, adaptability, low cost, and high spatial and temporal resolution. The ultrasound energy utilized has been proven to be safe and currently used in most medical environments, such as fetus, intravascular, indwelling, intracavitary, etc.

Current ultrasound transducer devices are typically comprised of a piezoelectric transducer, which sends and receives ultrasound, from which transformed ultrasound information is processed into real time images or other meaningful presentations, such as Doppler shift, tissue characterization, visualization of blood flow, etc. Over the years, ultrasound transducers have been incorporated into smaller devices, such as catheters disclosed in U.S. Pat. Nos. 5,325,860; 5,345,940; 5,713,363; 5,704,361, etc., by Seward et al., assigned to Mayo Foundation for Medical Education and Research, the common assignee of the present invention. In general, these catheters are thin tubes, which can be pushed into and manipulated within vessels or cavities. A transducer is disposed proximate a distal end of the catheter and generates underfluid images in the field of view. The shaft of these catheters is as large or larger than the transducer. The catheter is specifically designed to be manipulated by push/pull, using the torque of the catheter shaft and indwelling cables for tip articulation. Thus, the catheter is navigated through or within blood vessels, body cavities, and orifices, etc., and ultrasound functions as a visual substitute for visualizing the underfluid structure within the blood vessels, body cavities, and orifices, etc.

Characteristics of the surgical environment include need for sterility, small adaptable tools which can be incorporated into a surgical probe or finger, navigate very small spaces, unencumbered by cables, and do not cause injury to delicate tissues. Accordingly, it is desirable to have even smaller or miniaturized ultrasound transducers to meet the characteristics or needs of the surgical environment. Ultrasound transducers, for example, stand-alone, hand-held ultrasound transducers, are desired to be small enough to be easily accommodated within the confines of the surgical field, e.g. closed and/or confined spaces, small or delicate structures, etc.

### SUMMARY OF THE INVENTION

In accordance with this invention, the above and other problems were solved by providing a miniaturized ultrasound transducer (e.g. less than 4 mm×4 mm×10 mm) operable in small spaces, such as a blood-vessel-size type of underfluid structure. The ultrasound transducer may be mounted onto or incorporated into a holding device to be easily manipulated. The ultrasound transducer communicates with a processing unit via an electrical wire or cable or wirelessly.

In one embodiment of the present invention, a miniaturized ultrasound transducer operable in a blood-vessel-size type of underfluid structure includes an ultrasound transducer array and a transducer backing member. The miniaturized ultrasound transducer can be of any shapes, such as flat, round, or oval.

Still in one embodiment, the miniaturized ultrasound transducer may be mounted onto or incorporated into a holding device, such as a ring, flexible tube, cathether, ring, clamp, etc., to be easily manipulated.

Further in one embodiment, the ultrasound transducer has a size less than 4 mm in thickness/width and less than 10 mm in length, i.e. a transducer dimension of less than 4 mm (height)×4 mm (width)×10 mm (length).

Additionally in one embodiment of the present invention, the ultrasound transducer array can be arranged and configured into a linear, phased, sector, or a multidimensional array, which is capable of generating a multi-dimensional image.

Yet in one embodiment of the present invention, the ultrasound transducer is capable of having a working frequency in a range of 5 to 100 megahertz.

Still in one embodiment, the transducer backing member includes a suture tab having a suture hole(s). The suture tab allows the ultrasound transducer to be mounted onto and suture-secured to a structure.

Further in one embodiment, the transducer backing member includes a ring clip. The ring clip includes a pair of arms bendable towards and away from one another to allow the ultrasound transducer to be mounted onto a structure.

Additionally in one embodiment of the present invention, the ultrasound transducer includes a processing unit for sending and receiving ultrasound to and from the ultrasound transducer array.

Yet in one embodiment of the present invention, the ultrasound transducer further includes an electrical wire. The electrical wire is a very thin wire that connects to the ultrasound transducer array and to the processing unit.

Still in one embodiment of the present invention, the ultrasound transducer is a wireless device, wherein the ultrasound transducer array sends and receives ultrasound to and from the processing unit.

One of the advantages of the present invention is that it provides a standalone, hand-held ultrasound transducer that is small enough to navigate in small spaces, such as a blood-vessel-size type of underfluid structure. The ultrasound transducer may be mounted onto or incorporated into a holding device to be easily manipulated. One who performs an operation of the underfluid structure is able to self-manipulate the stand-alone, hand-held ultrasound transducer while performing the operation.

Another advantage of the present invention is that the miniaturized ultrasound transducer provides real time images of the underfluid structure and/or other meaningful presentations, such as Doppler shift, tissue characterization, visualization of blood flow, etc. of the underfluid structure.

These and various other advantages and features of novelty which characterize the invention are pointed out with particularity in the claims annexed hereto and form a part hereof. However, for a better understanding of the invention, its advantages, and the objects obtained by its use, reference should be made to the drawings which form a further part hereof, and to accompanying descriptive matter, in which there are illustrated and described specific examples of an apparatus in accordance with the invention.

### BRIEF DESCRIPTION OF THE DRAWINGS

Referring now to the drawings in which like reference numbers represent corresponding parts throughout:

FIG. 1A is a schematic view illustrating a front elevational view of a first embodiment of a miniaturized ultrasound transducer in accordance with the present invention.

FIG. 1B is a schematic view illustrating a side elevational view of the miniaturized ultrasound transducer shown in FIG. 1A.

FIG. 2A is a schematic view illustrating a front elevational view of a second embodiment of a miniaturized ultrasound transducer in accordance with the present invention.

FIG. 2B is a schematic view illustrating a side elevational view of the miniaturized ultrasound transducer shown in FIG. 2A.

FIG. 3A is a schematic view illustrating a front elevational view of a third embodiment of a miniaturized ultrasound transducer in accordance with the present invention.

FIG. 3B is a schematic view illustrating a side elevational view of the miniaturized ultrasound transducer shown in FIG. 3A.

FIG. 4A is a schematic view illustrating a front elevational view of a fourth embodiment of a miniaturized ultrasound transducer in accordance with the present invention.

FIG. 4B is a schematic view illustrating a side elevational view of the miniaturized ultrasound transducer shown in FIG. 4A.

FIG. 5A is a schematic view illustrating a perspective view of a fifth embodiment of a miniaturized ultrasound transducer in accordance with the present invention.

FIG. 5B is a schematic view illustrating a rear elevational view of the miniaturized ultrasound transducer shown in FIG. 5A.

### DETAILED DESCRIPTION OF THE PREFERRED EMBODIMENTS

In the following description of the illustrated embodiments, reference is made to the accompanying drawings that form a part hereof, and in which is shown by way of illustration several embodiments in which the invention may be practiced. It is to be understood that other embodiments may be utilized as structural changes may be made without departing from the spirit and scope of the present invention.

The present invention provides a miniaturized ultrasound transducer (e.g. less than 4 mm×4 mm×10 mm) operable in small spaces, such as a blood-vessel-size type of underfluid structure. The ultrasound transducer may be mounted onto or incorporated into a holding device to be easily manipulated. The ultrasound transducer communicates with a processing unit via an electrical wire or cable or wirelessly.

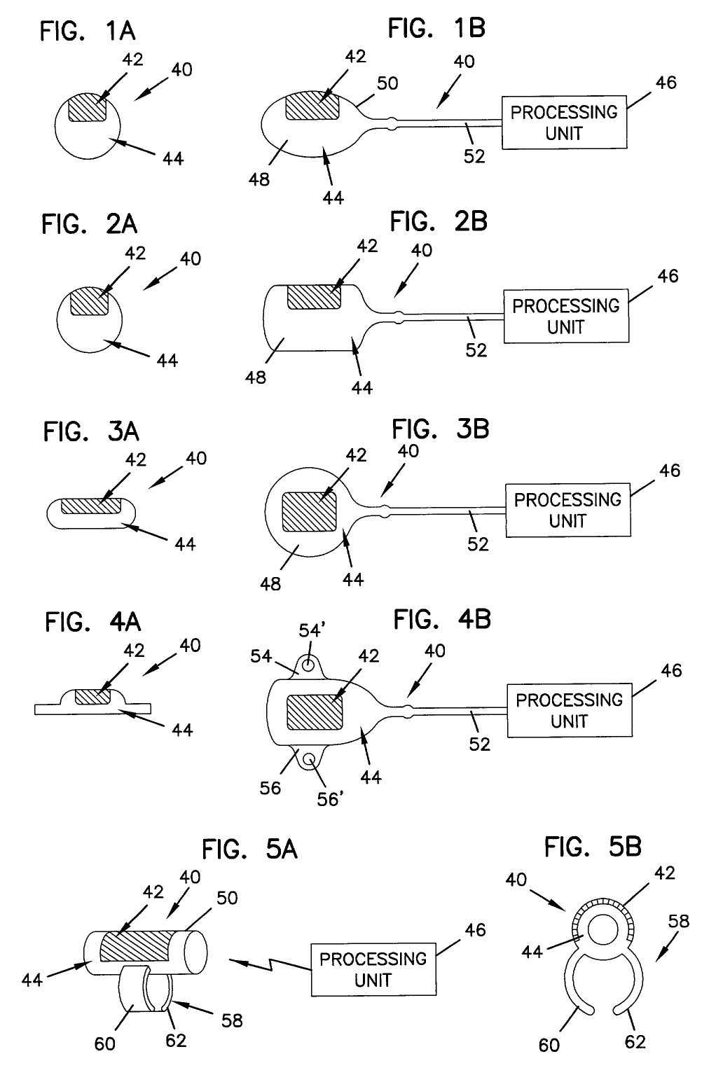

FIGS. 1A-B, FIGS. 2A-B, FIGS. 3A-B, FIGS. 4A-B, and FIGS. 5A-B illustrate five exemplary embodiments of a miniaturized ultrasound transducer 40. In FIGS. 1A-4B, the ultrasound transducer 40 includes an ultrasound transducer array 42, a transducer backing member 44, and a processing unit 46.

The miniaturized ultrasound transducer 40 may be mounted onto or incorporated into a holding device 58 as shown in FIGS. 5A-5B, such as a ring, flexible tube, cathether, ring, clamp, etc., to be easily manipulated.

The ultrasound transducer array 42 is mounted on an external surface 50 of the transducer backing member 44. An electrical wire 52, preferably a small, very thin flexible cable, is connected to the ultrasound transducer array 42 and to the processing unit 46.

The ultrasound transducer 40 can be arranged and configured in different shapes, such as a flat shape as shown in FIGS. 3A-3B, 4A-4B, a round shape as shown in FIGS. 1A-1B, 5A-5B, or an oval shape as shown in FIGS. 2A-2B, etc.

In FIGS. 4A and 4B, the transducer backing member 44 further includes a pair of suturing tabs 54, 56 having suturing holes 54′, 56′ which allow the ultrasound transducer 40 to be mounted or secured onto another structure (not shown), such as a probe, blood vessel, etc., via the suturing holes 54′, 56′.

In FIGS. 5A and 5B, the transducer backing member 44 further includes a holding device, such as a ring clip 58. The ring clip 58 includes a pair of arms 60, 62 bendable towards and away from one another to allow the ultrasound transducer 40 to be mounted onto a structure (not shown), such as a finger or a probe, etc.

It is appreciated that the miniaturized ultrasound transducer 40 can be arranged and configured in other shapes and/or constructions within the scope of the present invention. The miniaturized ultrasound transducer 40 is capable of functioning complete ultrasound attributes, such as Doppler, color flow imaging, parametrics, etc.

The ultrasound transducer 40 of the present invention has a size that is less than 4 mm in thickness/width and less than 10 mm in length, i.e. a dimension less than 4 mm×4 mm×10 mm.

The ultrasound transducer 40 has a working frequency in a range between 5 and 100 megahertz (MHz), and more preferably in a range between 5 and 30 megahertz (MHz). It is appreciated that the transducer may include other suitable frequency range within the scope of the present invention.

The ultrasound transducer array 42 can be arranged and configured into a linear, phased, sector, or a multi-dimensional array, which is capable of generating a multi-dimensional image.

Also as shown in FIGS. 5A and 5B, the ultrasound transducer 40 is a wireless device that sends and receives ultrasound to and from the processing unit 46.

Accordingly, the ultrasound transducer in accordance with the present invention are a new class of ultrasound transducers, for example, specifically tailored to applications used in surgical environment. The transducer is in a small size, e.g. less than 4 mm in thickness/width and less than 10 mm in length. Since the ultrasound transducer has such a small size and has very few attachments, the ultrasound transducer does not encumber a surgical field.

In use, the ultrasound transducer is an imaging device that can be attached to a surgical tool, clipped on a surgeon's finger, implanted within another surgical device, or accommodated in a specialty tool or device, etc. The transducer can function on or within body cavities, organs, tissues, orifices, or blood vessels, etc. One of the principal functions of the transducer is to substitute for conventional visualization of underlying structures. One of the other principal functions is to provide Doppler hemodynamics, color flow imaging, tissue characterization, and parametric imaging, etc. The resultant images can be 1-dimensional (1-D), 2-D, 3-D, or 4-D (including the motion dimension). The images can be displayed on a small consol, a hand-held device, or a wrist mounted screen, etc., that is suited for the specific circumstance. The transducer communicates with the processing unit via a small flexible wire or cable, or communicates with the processing unit wirelessly using wireless technology.

Having described the present invention in a preferred embodiment, modifications and equivalents may occur to one skilled in the art. It is intended that such modifications and equivalents shall be included within the scope of the claims which are appended hereto.

What is claimed is:

1. A miniaturized ultrasound transducer operable in a blood-vessel-size type of underfluid structure, comprising:
an ultrasound transducer array; and
a transducer backing member, the ultrasound transducer array being mounted and incorporated into the transducer backing member, wherein the ultrasound transducer is arranged and configured to be small enough to be operated in the blood-vessel-size type of underfluid structure.

2. The transducer of claim 1, wherein the ultrasound transducer has a dimension less than 4 mm (height)×4 mm (width)×10 mm (length).

3. The transducer of claim 2, wherein the ultrasound transducer array is arranged and configured into a linear array that is capable of generating a multi-dimensional image.

4. The transducer of claim 2, wherein the ultrasound transducer array is arranged and configured into a phased array that is capable of generating a multi-dimensional image.

5. The transducer of claim 2, wherein the ultrasound transducer array is arranged and configured into a sector array that is capable of generating a multi-dimensional image.

6. The transducer of claim 2, wherein the ultrasound transducer array is arranged and configured into a multi-dimensional array that is capable of generating a multi-dimensional image.

7. The transducer of claim 2, wherein the ultrasound transducer is in a flat shape.

8. The transducer of claim 2, wherein the ultrasound transducer is in a round shape.

9. The transducer of claim 2, wherein the ultrasound transducer is in an oval shape.

10. The transducer of claim 2, wherein the ultrasound transducer has a working frequency in a range of 5 to 100 megahertz.

11. The transducer of claim 2, wherein the transducer backing member includes a suture tab having a hole, the suture tab allows the ultrasound transducer to be mounted onto and suture-secured to a structure.

12. The transducer of claim 2, wherein the transducer backing member includes a ring clip, the ring clip includes a pair of arms bendable towards and away from one another to allow the ultrasound transducer to be mounted onto a structure.

13. The transducer of claim 1, further comprising a processing unit for sending and receiving ultrasound to and from the ultrasound transducer array.

14. The transducer of claim 2, further comprising a processing unit for sending and receiving ultrasound to and from the ultrasound transducer array.

15. The transducer of claim 13, further comprising an electrical wire that connects to the ultrasound transducer array and to the processing unit.

16. The transducer of claim 14, further comprising an electrical wire that connects to the ultrasound transducer array and to the processing unit.

17. The transducer of claim 1, wherein the ultrasound transducer is a wireless device that sends and receives ultrasound to and from the processing unit.

18. The transducer of claim 2, wherein the ultrasound transducer is a wireless device that sends and receives ultrasound to and from the processing unit.

19. The transducer of claim 13, wherein the ultrasound transducer is a wireless device that sends and receives ultrasound to and from the processing unit.

20. The transducer of claim 14, wherein the ultrasound transducer is a wireless device that sends and receives ultrasound to from the processing unit.
